(19) United States
(12) Patent Application Publication
(10) Pub. No.: US 2006/0276737 A1
(43) Pub. Date: Dec. 7, 2006

(54) Functional toe brace

(76) Inventor: Christopher Rose, Pittsburgh, PA (US)

Correspondence Address: CLIFFORD A. POFF, 9800B MCKNIGHT ROAD, SUITE 115, PITTSBURGH, PA, 15237, US

(21) Appl. No.: 11/143,894

(22) Filed: Jun. 2, 2005

(51) Int. Cl. A61F 5/00; A61F 13/06

(52) U.S. Cl. 602/30; 128/893

(57) ABSTRACT

A functional toe brace a thermally moldable substrate formed into a C-shaped cross sectional splint to restrain movement of the joint between the metatarsal and phalange foot bones of the great toe. The splint is secured to and positioned by a flexible retainer to traverse a foot sole orientated area and extent to a height laterally thereof to restrain movement between the metatarsal and phalange foot bones of the great toe. First and second elongate straps have ends secured by releasable fasteners, one to extend about the phalange foot bone of the great toe and the other to extend about the metatarsal foot bone of the great toe all for support by the foot. A U-shaped or an array of resilient standoff members is mounted on the foot sole orientated area to effective immobilization isolate the sesmoid bones and the attending ligaments in the area immediately to the sesmoid bones.

### CROSS-REFERENCE TO RELATED APPLICATIONS

Not applicable

### BACKGROUND OF THE INVENTION

1. Field of the Invention

The present invention relates to an appliance for providing engaged support of the foot for the joint between the metatarsal and phalange foot bones of the great toe and including, when desired, additional structure to immobilize the ligaments and surrounding soft tissue of the sesmoid bones underlying the great toe.

2. Description of the Prior Art

Turf toe, bone spurs, hallux valgus and bunions are well known disorders affecting a relatively localized area at the joint between the metatarsal and phalange foot bones of the great toe. Such disorders are common treated ailments usually including immobilizing the affected area for pain management. Turf toe, a common injury among athletes particularly football players, is a bruise at the metatarsal phalangeal joint which is located at the base of the great toe. The injury occurs when the Great toe is bent excessively backwards typically when the toe is jammed or other wise forced by contact with or against the ground upward from the plane containing the sole of the foot. The over extension causes damage by over stretching of the ligaments traversing the metatarsal phalangeal joint. The injury produces swelling and pain at the joint with notable pain and tenderness upon bending of the great toe upwards. Heretofore, the injury was treated with ice, a compression bandage, wearing a shoe to protect against bending of the affected joint and rest including the possible use of crutches to reduce the weight sustained by the foot. A persistence of such injuries can bring about the development of bone spurs in the area of the injury to the affected joint, although a development of bone spurs can be attributed to other causes.

While the onset of turf toe injuries is generally attributed a popular use of artificial Turf as the ground cover on football and soccer playing fields, bone spurs, hallux valgus and bunions are not necessarily an athletic related injury. U.S. Pat. No. 6,093,163 discloses a unitary device for the correction of hallux valgus made of an elastomeric fabric to enclose the forefoot circumferentially, and a smaller portion that encloses the great toe circumferentially. The elastomeric fabric is cut along a bias towards varus of the great toe to exert a varus force on the great toe, thus correcting the valgus deformity. U.S. Pat. Nos. 4,632,103 and 4,729,369 disclose bandage constructions generally using a connecting strip held at one end by a strap to encircle the central portion of the foot and the remaining end portion provided with strap segments having end portions connected by Velcro fasteners. The strap segments to be wrapped around the posterior aspect of the great toe are arranged to force the great toe outwardly and thereby relieve bunion. U.S. Pat. No. 6,159,173 discloses a forefoot bandage that is symmetric about a transverse axis and has two notches on opposite ends for positioning on the right and left foot. The notches and angled portions located along the bandage conform to the forefoot and during use one of the notches of the bandage is placed in the sulcus between the great toe and the second toe of the user and the bandage is then wrapped about the plantar region of the foot near the toes to support the joints of the great toe and forefoot. U.S. Pat. No. 6,238,359 discloses an insole insert to evert the foot with a raised area having inclined sloping edges at the first metatarsal area with support in the region of the ball of the foot and the first metatarsal head to evert the foot by raising the ball of the foot, thereby causing the foot to rotate outwardly, and treat a lower leg ailment commonly known as shin splints.

The known prior art fails to provide a structure to immobilize or at least provide immobilized support to restrict movements of the joint between the metatarsal and phalange foot bones of the great toe or, when desired, to immobilize or otherwise reduce the load bearing forces imposed on the ligaments and surrounding soft tissue of the sesmoid bones underlying the great toe.

Accordingly, it is an object of the present invention to provide a structure using a moldable splint carried by a flexible retainer for application to foot of a patient and provide immobilized support to restrict movements of the joint between the metatarsal and phalange foot bones of the great toe.

It is a further object of the present invention to provide a structure using a flexible retainer to support a moldable splint carrying spaced apart resilient supports for application to sole part of the foot of a patient to immobilize or otherwise reduce the load bearing forces imposed on the ligaments and surrounding soft tissue of the sesmoid bones underlying the great toe.

### SUMMARY OF THE INVENTION

More particularly, the present invention provides a functional toe brace comprising a thermally moldable substrate having a length and width sufficient to configure a molded C-shaped cross sectional splint to restrain movement of the joint between the metatarsal and phalange foot bones of the great toe, a flexible retainer engaged with the thermally moldable substrate having a splint support surface to traverse a foot sole orientated area and a height laterally thereof to restrain movement between the metatarsal and phalange foot bones of the great toe, a first elongate strap with releasable fastener extending from the flexible retainer to the phalange foot bone of the great toe for support by the great toe, and a second elongate strap with releasable fastener extending from the flexible retainer to the metatarsal foot bone of the great toe for support by the foot.

### BRIEF DESCRIPTION OF THE SEVERAL VIEWS OF THE DRAWINGS

The present invention will be more fully understood when the following description is read in light of the accompanying drawings in which:

FIG. 1 is a schematic bottom plan view of a patient's right foot showing the functional toe brace of the present invention applied thereto;

FIG. 2 is an isometric view of the functional toe brace of the present invention;

FIG. 3 is a prospective view of the functional toe brace shown in FIG. 2; and,

FIG. 4 is a sectional view taken along lines VI-VI of FIG. 3.

### DETAILED DESCRIPTION OF THE INVENTION

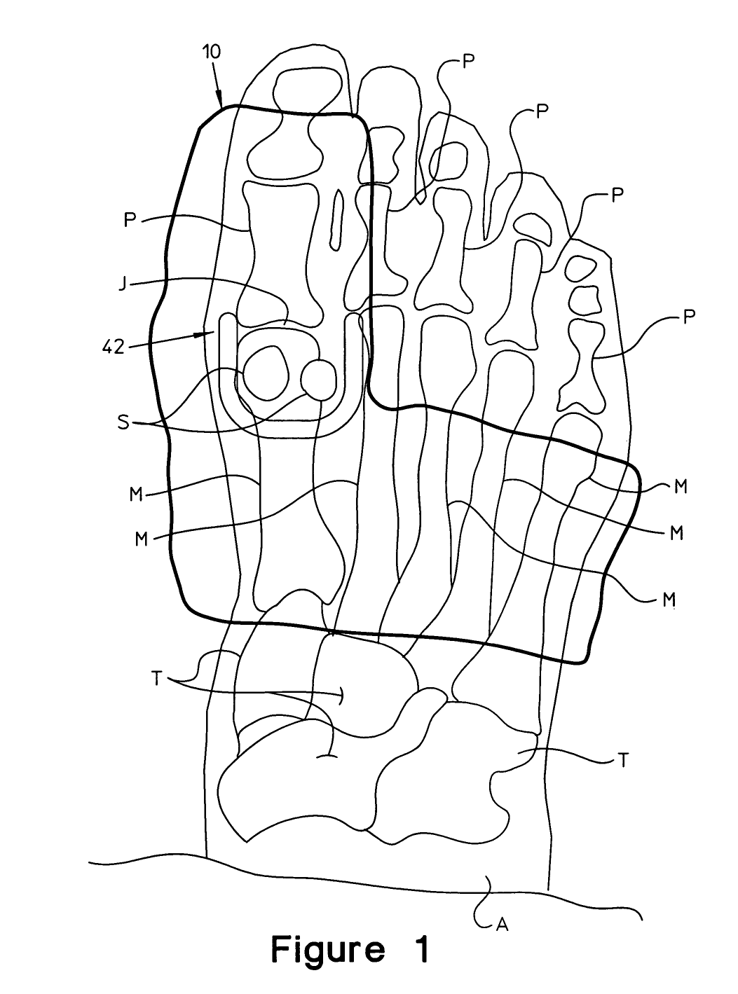

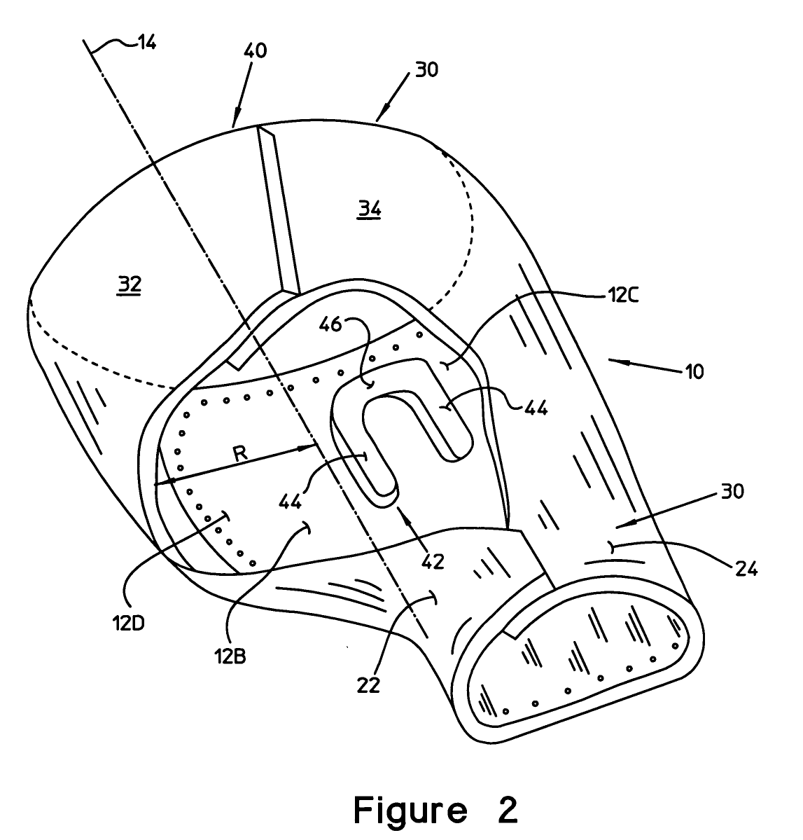

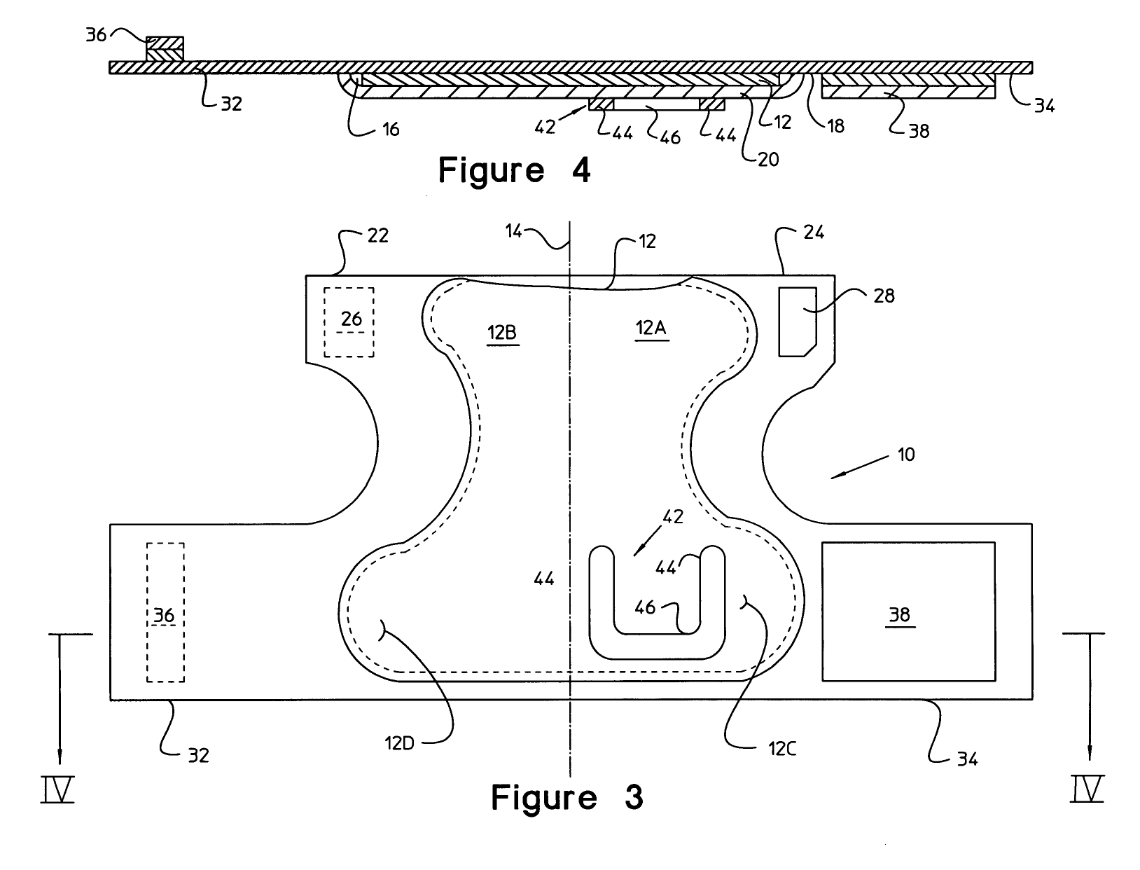

In FIG. 1 of the drawings, there is schematically illustrated the five metatarsals M forming joints J that interconnect the phalanges P to the Tarsal bones T extending to the ankle A. One aspect of the present invention is address a need to treat an injury to soft tissue particularly connecting with the sesmoid bones S as well as the sesmoid bones S, per se, located between the metatarsal M and phalange P of foot bones forming the great toe. The present invention provides a foot sprint 10 including a thermally moldable substrate 12 having a generally rectangular shape as shown in FIG. 3 generally half divided into a sole part 12A and side part 12B along a broken demarcation line 14 appearing in FIG. 3. The generally rectangular shape of the substrate 12 is modified by a protruding extension 12C to the sole part 12A and a protruding extension 12D to the side part 12B. The a length and width of the substrate is selected for a sufficiency to configure a molded C-shaped cross sectional splint as shown in FIG. 2 to restrain movement of the joint J between the metatarsal and phalange foot bones of the great toe. The sole part 12A and extension 12C provide a broad based surface area for immobilized support along the sole of the foot in the entire area traversing the joint J. The side part 12B and extension 12D also traverse the joint J of the great toe after molding by a generous radius R, shown in FIG. 2, to extend perpendicular to the sole part 12A and into custom configuration partly wrapping about the side of the foot and locating the extension 12D in an overlying position immediately above the joint J o the great toe. The arrangement is such that the sole part 12A and extension 12C of moldable substrate provides a support surface to traverse a foot sole orientated area and the side part 12B and extension 12D extends to a height laterally thereof to partly wrap the metatarsal and phalange foot bones of the great toe. The material selected to form the substrate 12 is per se well known in the art and preferably includes a thermosetting material that can be shaped after heating or a moisture curing resin shaped and cured to conform to the specific anatomical profile presented by the injured foot to be treated according to the present invention. The substrate is molded into a generally C-shaped cross sectional shape to produce a desired configuration for a functional toe brace to restrain movement of the joint between the metatarsal and phalange foot bones of the great toe

The substrate 12 with the predetermined shape and generally plainer profile is preferably secured in a compartment 16 formed by stitching together two layers of elastically flexible retainer fabric 18 and 20, each exhibiting sufficient elastic properties to accommodate the molded shaping of the substrate 12 in the compartment 16 while maintaining a long continued integrity to the attachment. The flexible retainer fabric 18 is preferably formed with extension straps 22 and 24 having overlapping and confronting face surfaces to which rectangular segments of hook and pile fasteners 26 and 28, respectively, are secured. The extension strips form a first elongate strap 30 used to establish an encircled relation and exhibit the flexible properties of the retainer fabric 18 and thereby provide by the rigidity of the molded substrate as the basis for restrained support against movement by the great toe. Preferably, the length of the extension strips 22 and 24 are such that the hook and pile fasteners 26 and 28 reside along a generally upward facing surface of the great toe. The flexible retainer fabric 18 is also preferably formed with extension straps 32 and 34 having secured to overlapping and confronting face surfaces rectangular segments of hook and pile fasteners 36 and 38, respectively, forming a second elongate strap 40 for completing an encircling relation by the flexible retainer fabric 18 for encircling the foot generally about the site of the five metatarsals M. In view of the forgoing description and the illustration by the drawings, the functional toe brace of the present invention is effective in this aspect of providing a structure using a moldable splint carried by a flexible retainer for application to foot of a patient to provide a desired immobilized support to restrict movements of the joint between the metatarsal and phalange foot bones of the great toe.

A further feature of the functional toe brace of the present invention is the inclusion on the moldable splint and used also to support a resilient standoff for application to sole part of the foot of a patient to immobilize or otherwise reduce the load bearing forces imposed on the ligaments and surrounding soft tissue of the sesmoid bones underlying the great toe. For this purpose, as shown in FIGS. 2-4, there is provided an elastically compressible generally resilient standoff 42 secured by a layer of adhesive to the exposed surface of the flexible retainer fabric 18 or, if desired, to the underlying surface of the substrate 12. The location of the resilient standoff 42 upon the surface area of the sole part 12A and extension 12C is chosen to isolate the sesmoid bones and the attending ligaments in the area immediately to the sesmoid bones. Effective immobilization at the designated site is provided by a standoff having a generally U-shaped configuration with the parallel legs 44 extending in a generally parallel relation with the metatarsal M of the great toe at opposite lateral sides of the sesmoid bones S. Additionally, the bridging section 46 of the U-Shaped standoff traverses the longitudinal extending ligaments rearward of the metatarsal and phalange foot bones of the great toe. The uncompressed height of the resilient standoff is at least ⅛ inch to effectively isolate the area containing the sesmoid bones and surrounding soft tissue of the attending ligaments. The material for providing resiliency to the standoff is commonly available and used to isolate soft tissue under a load generally comprised of the body weight. The resiliently is also effected t confirm the surface contour to the surface contour encountered in the relevant sole area of the foot. Another configuration of the standoffs takes the form of an array of discreet raised ribs arranged like the legs and the bridge sections of the U-Shaped standoff to function in substantially the same manner.

While the present invention has been described in connection with the preferred embodiments of the various figures, it is to be understood that other similar embodiments may be used or modifications and additions may be made to the described embodiment for performing the same function of the present invention without deviating therefrom. Therefore, the present invention should not be limited to any single embodiment, but rather construed in breadth and scope in accordance with the recitation of the appended claims.

1. A functional toe brace comprising:
a thermally moldable substrate having a length and width sufficient to configure a molded C-shaped cross sectional splint to restrain movement of the joint between the metatarsal and phalange foot bones of the great toe;
a flexible retainer engaged with said thermally moldable substrate having a splint support surface to traverse a foot sole orientated area and a height laterally thereof to restrain movement between the metatarsal and phalange foot bones of the great toe;
a first elongate strap with releasable fastener extending from said flexible retainer to the phalange foot bone of the great toe for support by the great toe; and
a second elongate strap with releasable fastener extending from said flexible retainer to the metatarsal foot bone of the great toe for support by the foot.

2. The functional toe brace according to claim 1 wherein said flexible retainer comprises two layers of elastically flexible retainer fabric stitching together and forming a compartment for housing said thermally moldable substrate.

3. The functional toe brace according to claim 1 wherein said thermally moldable substrate comprises a generally rectangular generally rectangular shape to form a sole part for immobilized support along the sole of the foot in the entire area traversing the joint between the metatarsal and phalange foot bones of the great toe.

4. The functional toe brace according to claim 3 wherein said generally rectangular generally rectangular shape of said thermally moldable substrate further comprises a side portion generally perpendicular to said sole portion and into custom configuration partly wrapping about the side of the foot in a overlying position immediately above the joint between the metatarsal and phalange foot bones of the great toe.

5. The functional toe brace according to claim 1 further including a resilient standoff supported by said a foot sole of said splint support surface to reduce the load bearing forces imposed on the ligaments and surrounding soft tissue of sesmoid bones underlying the great toe.

6. The functional toe brace according to claim 1 wherein said resilient standoff is further defined by having parallel legs extending along said foot sole in a generally parallel relation with the metatarsal of the great toe at opposite lateral sides.

7. The functional toe brace according to claim 6 wherein said resilient standoff is further defined by having a bridging section traverse to said parallel legs on said foot sole rearward of the metatarsal and phalange foot bones of the great toe.

8. The functional toe brace according to claim 7 wherein said resilient standoff is further defined by having an uncompressed height of at least ⅛ inch on said foot sole to effectively isolate an area containing sesmoid bones and surrounding soft tissue of the attending ligaments.

9. The functional toe brace according to claim 6 wherein said resilient standoff is defined by having a continuous generally U-shaped configuration comprised of said parallel legs and said bridging section.

10. The functional toe brace according to claim 7 wherein said resilient standoff is adhesively attached to said foot sole.
